(19) United States
(12) Patent Application Publication
(10) Pub. No.: US 2018/0343898 A1
(43) Pub. Date: Dec. 6, 2018

(54) DISINFECTING CONVEYOR SYSTEM

(71) Applicant: Radiant Industrial Solutions LLC, Houston, TX (US)

(72) Inventors: Mohammed Alzeer, Riyadh (SA); Tim Bartlett, Houston, TX (US); Drew Jaworski, Houston, TX (US); Michael R. Guerrette, Houston, TX (US); Troy Smith, Houston, TX (US)

(21) Appl. No.: 16/053,673

(22) Filed: Aug. 2, 2018

Related U.S. Application Data
(63) Continuation-in-part of application No. PCT/US17/16272, filed on Feb. 2, 2017.
(60) Provisional application No. 62/289,990, filed on Feb. 2, 2016.
(60) Provisional application No. 62/662,884, filed on Apr. 26, 2018.

(51) Int. Cl. A23L 3/28; A61L 2/10; A61L 2/26

(52) U.S. Cl. CPC A23L 3/28; A61L 2/26; A61L 2/10

(57) ABSTRACT

A disinfecting conveyor system utilizes ultraviolet light to disinfect a product as it passes through an enclosure on a conveyor. A UV light sensor provides feedback to a control system that can change the amount of UV light exposure, speed of conveyor, and disinfection reaction. A UV light sensor may be a coupled to the conveyor and the UV light intensity and time of exposure within the enclosure may be used to calculate a UV light dose. A user of the system may input a required UV light dose or the type of product to be disinfected and the control system may automatically control the speed of the conveyor and/or the UV light intensity to provide the required UV dose. The system may utilize an air filtration and/or cooling system to provide a flow of clean and cool process air to the enclosure to maintain a temperature that is below a threshold temperature.

### CROSS REFERENCE TO RELATED APPLICATIONS

This application is a continuation in part of PCT/US2017/016272, filed on Feb. 2, 2017 which claims the benefit of priority to U.S. provisional patent application No. 62/289,990 filed on Feb. 2, 2016, and this application also claims the benefit of priority to U.S. provisional patent application No. 62/662,884, filed on Jun. 8, 2018; wherein all applications are entitled Disinfecting Conveyor System; and wherein the entirety of all prior applications are hereby incorporated by reference herein.

### BACKGROUND OF THE INVENTION

### Field of the Invention

This invention relates to the conveyance or transport through conveyor systems for disinfecting products including foods, containers, or food touch materials as they pass through the conveyor. The objective is to create a sanitizing environment where the food, container, or product is not altered by the intervening technology.

### Background

Conveyor systems for disinfecting products, including packaging products, containers, lids, bottles, consumer products, toys and food products, can overheat, dry, or change the properties of the products, resulting in a critical property change or increase bacteria or pathogen growth or an undesirable change in the product due to exposure to elevated temperatures as well as over exposure to technology intervention. For example, a bread product moving through a disinfecting conveyor may be heated from an ultraviolet (UV) light source, thereby creating on increasing bacteria and or changing the quality texture, dryness of the bread. In addition, bacteria may grow on components of the conveyor system, such as the conveyor belt and the bacteria may be transferred to other products as they move through the conveyor. There exists a need for a disinfecting conveyor system that can maintain temperature below a threshold temperature within the enclosure while performing the disinfection duty at the correct and measured level of exposure with adjustment to insure the product is safe and disinfected without adverse conditions.

### SUMMARY OF THE INVENTION

The invention is directed to a disinfecting conveyor system and/or sanitizing, complete irradiation of microbes, system that enables a controlled dose of UV light to be provided to a product at a controlled environment, wherein the temperature humidity and/or gas exposure is controlled. In addition, an exemplary disinfecting conveyor system provides a flow of filtered air into the disinfection enclosure. An exemplary disinfecting conveyor system comprises a control system that monitors the UV light intensity and/or dose of UV light as well as temperature within the disinfecting enclosure. An air filtration system to remove particles from inlet air and an air cooling system to cool the inlet air and provide a cooled flow of process air in order to maintain the temperature within the enclosure below a threshold temperature. In an exemplary embodiment, the disinfecting conveyor system is configured to meet FDA title 21 standard, wherein the system destroys all pathogens but not necessarily all viruses. The current FDA title 21 standard, as of filing of this application, is hereby incorporated by reference herein.

An exemplary disinfecting conveyor system comprises a conveyor that extends through an enclosure. The conveyor has a receiving end and a deliver end. Product is received on the receiving end of the conveyor, passes through the enclosure where it receives a dose of UV light, and is removed from the conveyor on the delivery end. A conveyor may extend completely through an enclosure and extend out from inlet and/or outlet end of the enclosure. A conveyor may comprise a conveyor belt that spins or rotates to move a product through the enclosure. A conveyor belt may comprise a plurality of apertures to enable UV light to pass therethrough. A conveyor belt may comprise apertures that make up a substantial portion of the conveyor belt surface, wherein the aperture conveyor belt has a percent open area of at least about 50%, or more preferably at least about 75%, and even more preferably at least about 90 or more. A large percentage open area will enable more of a product resting on the conveyor belt to be exposed to UV light as it passes through the disinfecting conveyor system.

A conveyor belt may be plastic or metal and is preferably a surface the prevents microbial formation. For example, the conveyor belt, or portion thereof may be a metal having apertures or a metal net, mesh or woven screen material to allow UV light to pass therethrough. A metal conveyor belt may be steel, stainless steel, aluminum, copper, and may consists of or comprise a metal that is natural antimicrobial including, but not limited to, titanium, cobalt, nickel, copper, zinc, zirconium, molybdenum, tin, and lead. A conveyor belt may be made out of a plastic, and preferably a plastic that is resistant to degradation from UV light exposure including, but not limited to Acetal homopolymer, Nylon, Polybutylene terephthalate (PBT), polyethylene (PE), high density polyethylene (HDPE), polyvinylidene fluoride (PVDF), polyetherimide (PEI), polyozymethylene (POM), fluoropolymers, Fluorinated ethylene propylene (FEP), THV (tetrafluoroethylene, hexafluoropropylene and vinylidene fluoride), perfluoroalkoxy alkane (PFA), or blend or composites of these materials and the like. Again, a conveyor belt, or portion thereof may be made out of plastic that comprises apertures therethrough, such as a plastic net or woven plastic material. A belt may have a support structure and then be coated with a UV resistant polymer. For example, a belt may be a metallic screen or net that is coated with Acetal, or nylon for example.

An exemplary disinfecting conveyor system comprises a plurality of UV light sources configured to project UV light onto a product as it moves through the enclosure. UV light may be provided by elongated bulbs, typically cylindrical tubes, that emit the UV light over an elongated length of the bulb, such as at least about 10 cm or more, or 25 cm or more, or 50 cm or more and any range between and including the lengths provided. A UV light source may be a light emitting diode, (LED), and a plurality of LED lights may be configured in a single light fixture. UV light is used to kill or inactivate microorganisms by destroying nucleic acids and affecting their DNA. UV light can interfere with an organisms' metabolic system and reproductive processes, rending the organism no longer pathogenic. UV light can be used for destroying pathogens or organisms including, but not limited to molds, bacteria and viruses. The dosage recommended to destroy and/or deactivate the organism varies. Table 1 provides a list of organisms and exemplary dosage levels recommended for deactivation by UV light. This dosage level may not be required however. In addition, listeria is another pathogen that may be deactivated by the system described herein. Note that the amount of light required may depend on the spectrum of light used and the wattage. Light at a wattage of 10 to as much as 2000 watts or more may be used in the disinfecting conveyor system.

TABLE 1

| | *Dosage required |
|---|---|
| | to deactivate |
| Pathogen | μw/cm² |
| Mold | |
| Aspergillus flavus | 99,000 |
| Aspergillus niger | 330,000 |
| Bacteria | |
| Bacillus anthracis | 8,700 |
| Bacillus anthracis spore | 146,200 |
| Escherichia coli | 1,840 |
| Legionella pneumophila | 12,300 |
| Mycobacterium tuberculosis | 3,240 |
| Salmonella typhi-Typhoid Fever | 7,000 |
| Staphylococcus aureus (MRSA) | 1,990 |
| Vibrio comma-Cholerae | 6,500 |
| Clostridium difficile-C-diff | 38,380 |
| Vancomycin Resistant Enterococci (VRE) | 18,700 |
| Virus | |
| Infectious Hepatitis | 8,000 |
| Influenza A | 6,600 |

*99.99% deactivation

UV light spans wavelengths from 400 nm to 10 nm. UV light sources that produce UV light with wavelengths of 200 to 280 nm may be used for destroying microorganism and UV light in the range of about 250 nm to 280 nm may be particularly effective for disinfection applications. In an exemplary embodiment, a plurality of UV light sources are configured to produce UV light substantially 360 degrees about the product as it moves through the enclosure. As a product moves through the enclosure, it may be effectively exposed to UV light whereby substantially the entire outer surface of the product is exposed to UV light, wherein at least about 90% of the outer surface is exposed to UV light directly or indirectly, such as through reflected light. The conveyor belt may be UV light translucent and a UV light source under the belt may project UV light through the belt and onto the product. The plurality of UV light sources may be configured in any suitable location within the enclosure and in an exemplary embodiment, at least some of the UV light sources are configured above the top surface or top span of the conveyor belt and at least some of the UV light sources are configured below the top span of the conveyor belt. UV light sources may be configured below the top span of the conveyor belt and above the bottom span or return span of the conveyor belt, or below the bottom span or return span of the conveyor belt. In addition, reflective materials may be configured to reflect UV within the enclosure, such a titanium dioxide (TiO2) that may be configured on the interior walls of the enclosure or on a conveyor belt and the coated with a UV transparent plastic, for example. The UV light sources may be any suitable power level and may vary depending on the number of sources configured within the enclosure. UV light sources having an output of 5 W or more, 10 W or more, 50 W or more may be used in the disinfecting conveyor system.

An exemplary conveyor belt is UV light translucent or transparent and an exemplary disinfecting conveyor system comprises a UV light source above the top surface of the conveyor belt and a UV light source below the top surface of the conveyor belt. Food or other products are transported through the disinfecting conveyor system of the top surface of the belt and may be exposed to UV light from the top UV light source and the bottom UV light source. In this exemplary embodiment, a product portion of the belt is translucent to enable UV light treatment of substantially the entire product passing therethrough. An exemplary UV transmission belt may have an edge support comprising a reinforcement that extends along the edge of the belt. An exemplary edge support may include a folded portion of the belt and may include a support material, such as a fabric that is attached to the belt. An exemplary edge support may extend a limited portion across the width of the belt, such as no more than about 5 mm, no more than about 10 mm, no more than about 25 mm, no more than about 50 mm and any range between and including the values provided. Put another way, the product portion of the belt may extend a substantial portion of the belt width such as at least about 75% of the width or more, about 85% of the width or more. An exemplary support material may be a woven fabric, such as a woven fiberglass, or a woven fluoropolymer. In some embodiments, a support material may extend across the product portion of the belt when the support material is UV translucent thereby creating no to very little shadowing of UV light on the product. The support material may prevent stretching of the belt, since the product portion, or center span may consist essentially of or consist of a UV translucent or transparent material, such as a plastic or polymer film that may be susceptible to stretching over time. An exemplary UV transmission belt may comprise, consist essentially of, or consist of a material that is at least UV translucent including, but not limited to glass, or a plastic such as polyethylene (PE), high density polyethylene (HDPE), polyvinylidene fluoride (PVDF), polyetherimide (PEI), polyozymethylene (POM), fluoropolymers, Fluorinated ethylene propylene (FEP), THV (tetrafluoroethylene, hexafluoropropylene and vinylidene fluoride), perfluoroalkoxy alkane (PFA), or blend or composites of these materials and the like. A fluoropolymer material may be preferred as is not as susceptible to degradation from exposure to UV light as other plastics and polymers. An exemplary UV transmission belt or a product portion of said belt may be at least UV translucent wherein about 25% or more of the UV light passes through the belt and preferably about 50% or more and even more preferably about 85% or more. When the belt has about 90% of more UV light transmission it is UV transparent for the purposes of this invention. The light transmission, such as UV light transmission may be checked using a photometer to measure the amount of UV light incident on a first surface and to measure the amount that passes through the belt. ASTM D1746-15 may be used to measure the light transparency generally through the belt; this test method is incorporated by reference herein. An exemplary UV transmission belt may be a sheet or solid layer of plastic or may comprise apertures therethrough.

An exemplary disinfecting conveyor system comprises a belt tracking system which may incorporate belt location sensors that provide an input to the control system and a device to change the direction of the belt to keep the belt aligned through the conveyor. The edge support portion may be thicker than the product portion of the belt and this increased thickness may be used on rollers to keep the belt aligned. A portion of the belt may comprise a belt tracking feature such as a printed feature that is detected by the belt location sensor or holes in the belt that are detected by the belt location sensor. The belt tracking feature may be configured along the edges, such as in the edge support portion.

An exemplary disinfecting conveyor system comprises a UV light sensor configured within the enclosure to measure the intensity of the UV light within the enclosure. One or more stationary UV light sensors may be configured within the enclosure and provide UV light intensity measurements to a control system. The control system may change the number of UV lights that are operating, or the power level to the UV lights in order to maintain a desired or input UV light intensity level. In an exemplary embodiment, a disinfecting conveyor system comprises a conveyor UV light sensor that is coupled with the conveyor and monitors the UV light intensity as it moves through the enclosure. The measured UV light intensity as a function of time may be used to calculate a UV exposure dose for a product that moves through the enclosure. Different products may require different doses of UV to ensure effective disinfection. For example, meat products may require a higher dose of UV light than bread. A user may input the dose requirement or type of food product for disinfecting through a user input and the control system may modify the speed of the conveyor and the UV light intensity produced by the plurality of UV lights to ensure that this threshold UV dose level is achieved.

An exemplary disinfecting conveyor system comprises a user input that enables a user to input a UV light intensity value, a UV light dose or the type of material to be treated with the UV, i.e., meats, dry goods, vegetables, packaging, etc. The control system of the disinfecting conveyor system may then receive inputs such as a measured UV light intensity, speed of the conveyor, and the like, and use these readings to change one or more settings of the conveyor system. For example, a user may input a desired dose of UV for a product and the control system may utilize a measured UV light intensity value and the speed of the conveyor to determine the dose that will be provided, or may set the UV light intensity or speed of the conveyor to ensure that the product receives the required dose. An algorithm or computer program within the control system may also allow a user to set the dose and speed, within limits, and the control system may determine the number and/or intensity of the UV lights required to achieve the required UV light dose. The controller may alert the user if they try to set a speed that is too high, wherein the product would not receive an effective UV light dose even with the UV lights on to a maximum set point. In another embodiment, a control system may receive input that the UV light intensity is below an input UV light intensity value, due to one or more UV light sources going out, and may then turn on additional UV light sources or increase the intensity of UV light produced by the remaining UV light sources. A warning signal may be generated by the control system, such as a flashing light or alarm sound, to indicate that the disinfecting conveyor system is not performing to input set points, such as UV light intensity being below or above a set point value.

An exemplary disinfecting conveyor system comprises an air filtration system configured to remove particles from inlet air and deliver a flow of clean air as a process air into the enclosure. An air filtration system may be configured to be HEPA efficient thereby removing 99.7% of 0.3 μm or larger particles, or ULPA efficient removing 99.999% of 0.1 μm or larger particles from the inlet air flow. In an exemplary embodiment, the filtration system removes at least 99.95% of 0.3 μm or larger particles from the inlet air. The clean air may be subsequently introduced into the air cooling system 80 or these systems may be reversed having the inlet air cooled prior to being filtered. An inlet air duct may be configured to extend through a portion of the enclosure and be exposed to the UV light. An inlet air duct may be made out of material that is translucent or preferably transparent to UV light, thereby exposing the inlet air to the UV light for the purposes of destroying any microorganism. An air inlet duct may be made out of UV transparent plastic or a ceramic, such as glass, for example.

An exemplary disinfecting conveyor system comprises an air cooling system configured to cool the inlet air to produce a cooled air flow for deliver as process air into the enclosure. Products, such as food products, may require that the temperature be maintained below a threshold temperature to reduce the potential of bacteria growing and/or to prevent undesirable cooking of the food. A temperature sensor within the enclosure may monitor the temperature and provide an enclosure temperature reading to a control system that regulates the amount and/or temperature of the process air entering the enclosure. The control system may be a part of the air cooling system or a separate control system. An exemplary control system has a user input feature to allow a user to set the threshold temperature or UV dose required for a product. The exemplary disinfecting conveyor system of the present invention may be used for food products during processing or harvesting of food products, wherein food products, such as fruits or vegetables are conveyed during a harvesting operation or between harvesting and a production facility. An exemplary disinfecting conveyor system may be used for transport of a product from one location to another and the conveyor belt may be a loop that is driven to transport or convey the product from the inlet to the outlet. In another embodiment, a shaker table may be coupled with the conveyor system and the product may move from the inlet to the outlet of the disinfecting conveyor system by vibration.

An exemplary disinfecting conveyor system comprises air inlet vents that provide air flow into the enclosure and these may be configured to provide a uniform and laminar flow of air over the product. A laminar flow of air may prevent unwanted stirring of the air or removal of debris from the product as it passes through the conveyor. An air inlet vent may be an air-knife that produces a laminar flow of air. In a preferred embodiment, the air inlet vents are configured above the conveyor and produce a flow of air that flows down over the product and into air exhaust vents that are configured below the conveyor. In this way, any debris will be removed from the enclosure by the flow of air from top to bottom. The air exhaust vents may comprise a trap to collect any debris. Process air may be recirculated to the air filtration and cooling system.

An exemplary disinfecting conveyor system comprises a replaceable plastic or quartz liner that extends around a portion of the conveyor and within the enclosure and is UV transparent. A replaceable liner may ensure that bacteria or other microorganism that may grow within the enclosure and debris from the enclosure and/or conveyor drive system from contaminating product. A replaceable liner may have apertures for the UV light sources and/or for the air flow into or out of the enclosure. In addition, a separator may be configured over a portion of the conveyor, such as over the top surface of the conveyor to prevent any debris, such as a ruptured UV light bulb, from falling down onto the conveyor or product. A separator may be configured only under the UV light sources or may extend over the top surface of the conveyor and may be UV light translucent or UV light transparent and may be permeable to allow air flow through the separator. It may be preferred that the separators only extend under the light sources to allow flow of cooling air down onto the produce without being impeded by a separator. This may be important to produce a laminar flow down over the product.

The process air may be controlled to a set or desired humidity level. It may be preferred to reduce the humidity level of the process air, such as through the air conditioning system which comprises a dehumidification attribute, or a designated dehumidification unit. It may be desirable to reduce the humidity down to about 50% or less, or about 30% or less, or about 10% or less. Low humidity may prevent growth of bacteria. In addition, a gas may be introduced into the conveyor system, such as an inert gas including nitrogen, for example, or any other suitable inert gas. This may also reduce the bacteria growth as it reduces the oxygen level.

The summary of the invention is provided as a general introduction to some of the embodiments of the invention, and is not intended to be limiting. Additional example embodiments including variations and alternative configurations of the invention are provided herein.

### BRIEF DESCRIPTION OF THE DRAWINGS

The accompanying drawings are included to provide a further understanding of the invention and are incorporated in and constitute a part of this specification, illustrate embodiments of the invention, and together with the description serve to explain the principles of the invention.

FIG. 1 shows a perspective view of an exemplary disinfecting conveyor system comprising a conveyor that extends through an enclosure having filtered and cooled air.

FIG. 2 shows a side view of an exemplary disinfecting conveyor system comprising a conveyor belt having apertures therethrough to enable the UV light to be incident on the product from the top and bottom.

FIG. 3 shows a graph of an output from a conveyor UV sensor.

FIG. 4 shows an inlet end of an exemplary disinfecting conveyor having a receiving end of the conveyor belt.

FIG. 5 shows a top view of an exemplary conveyor belt that is a UV transmission belt having a product portion that is at least UV translucent.

FIG. 6 shows a cross sectional view of an exemplary conveyor belt that is a UV transmission belt having a product portion that is at least UV translucent and an edge portion that is thicker.

### DETAILED DESCRIPTION OF THE ILLUSTRATED EMBODIMENTS

Corresponding reference characters indicate corresponding parts throughout the several views of the figures. The figures represent an illustration of some of the embodiments of the present invention and are not to be construed as limiting the scope of the invention in any manner. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Certain exemplary embodiments of the present invention are described herein and are illustrated in the accompanying figures. The embodiments described are only for purposes of illustrating the present invention and should not be interpreted as limiting the scope of the invention. Other embodiments of the invention, and certain modifications, combinations and improvements of the described embodiments, will occur to those skilled in the art and all such alternate embodiments, combinations, modifications, improvements are within the scope of the present invention.

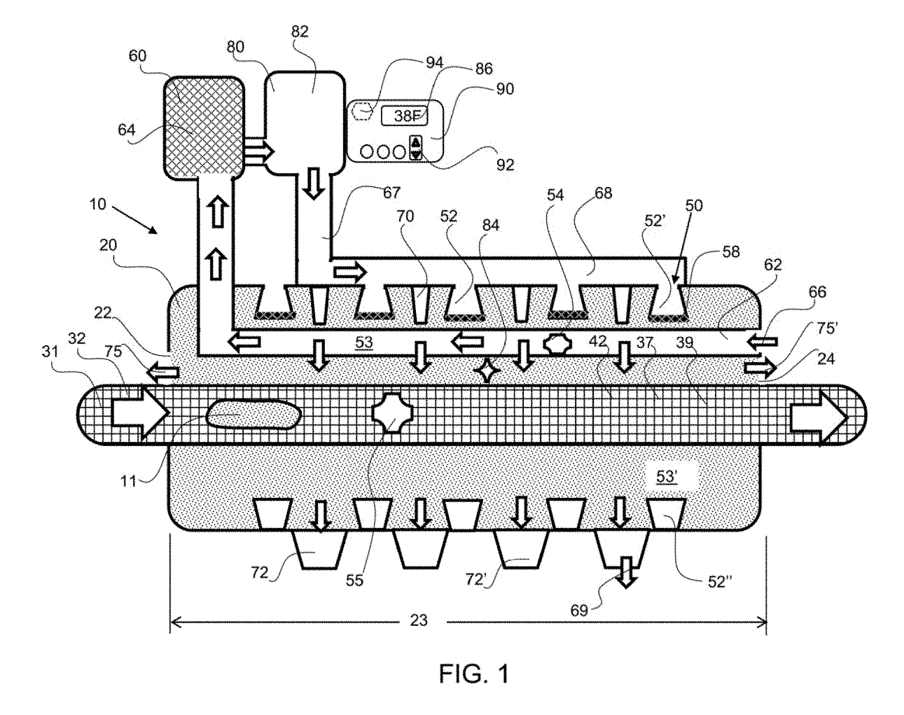

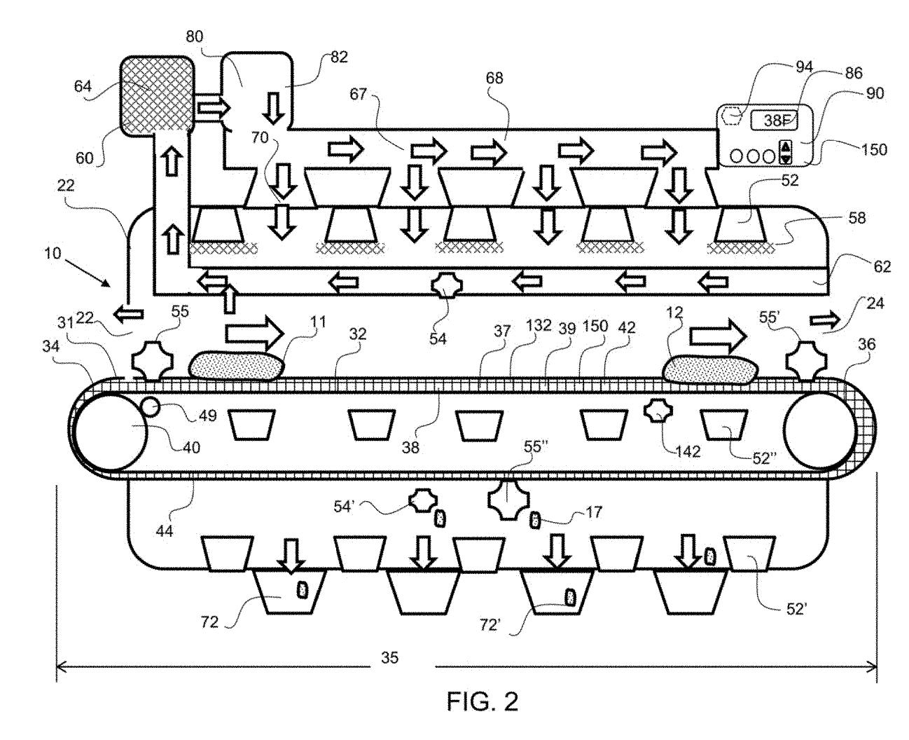

As shown in FIG. 1, an exemplary disinfecting conveyor system 10 comprises a UV disinfecting system 50 configured to disinfect product moved through an enclosure 20 on a conveyor 30. The exemplary conveyor 30 comprises a belt 32 that extends through the enclosure and has a length 23 from an inlet 22 to the outlet 24. A product 11, such as a food product, may traverse through the enclosure on the conveyor 30 having a conveying surface 31 of the conveyor belt 32 and receive exposure to UV light 53 that effectively disinfects the product. The conveying surface 31, is the top surface 37 of the conveyor belt that transports the product from the inlet 22 to the outlet 24 of the enclosure and comprises apertures 39 therethrough to allow UV light from the UV light sources 52, 52″, above and below the top span 42, respectively to pass through the conveyor. As discussed herein, the bottom UV light sources may be between the top span and bottom span 44, as shown in FIG. 2, of the conveyor. A plurality of UV light sources 52-52″ are configured to expose substantially the entire outer surface of the product to UV light. The UV light sources 52′ may project through the bottom span and the top span and may also disinfect the conveying surface of the conveyor belt with the product 11 removed. A plurality of UV light sources are configured above the conveyor belt and a plurality of UV light sources are configured below the top span of the conveyor belt and may be below the bottom span of the conveyor. UV light may cause an undesirable rise in temperature of the product and the disinfecting conveyor system provides a flow of cool air, or process air 68 into the enclosure to maintain a temperature below a threshold temperature. A control system 90, having a user input feature 92 and a microprocessor 94, allows a user to input a threshold temperature. As described herein the threshold temperature may be different for different types of products that are disinfected. The control system may run a program or algorithm to determine the speed and/or light intensity required to provide an effective UV light dose. An exemplary air cooling system 80 comprises an air-conditioner 82 to cool inlet air 66 and a temperature sensor 84 configured to measure the temperature within the enclosure and provide feedback to the control system. The flow and/or temperature of the process air 68 may be automatically controlled by the control system based on the temperature sensor reading and the input threshold temperature 86.

The exemplary disinfecting control system further comprises an air filtration system 60 comprising air filters 64 that remove particles from the inlet air 66 to produce clean air 67. An air filtration system may be configured to be HEPA or efficient thereby removing 99.7% of 0.3 μm or larger particles, or ULPA efficient removing 99.999% of 0.1 μm or larger particles from the inlet air flow. In an exemplary embodiment, the filtration system removes at least 99.95% of 0.3 μm or larger particles from the inlet air. The clean air 67 may be subsequently introduced into the air cooling system 80 or these systems may be reversed having the inlet air cooled prior to being filtered. The process air 68 is introduced into the enclosure through air inlet vents 70. The process air flows down over the product and into air exhaust vents 72. This top to bottom flow of process air ensures that any debris from the product will be pushed down and out of the enclosure to prevent bacteria from forming within the enclosure. In an exemplary embodiment, a positive flow of air 75, 75′ flows out of the enclosure from the inlet 22 and outlet 24, respectively, and prevents debris from entering into the enclosure. Curtains or other closures may be configured to dose or partially dose the inlet and/or outlet to prevent debris from entering. The exhaust air 69 may be recirculated back to the air filtration and/or air cooling system.

As shown in FIG. 1, the inlet air 66 is drawn into an inlet air duct 62 that extends within the enclosure 20 and is exposed to the UV light 53 from the UV light sources 52. This exposure to the UV light source treats and disinfects the inlet air prior to being introduced into the air filtration and air cooling systems. Exposing the inlet air to the UV light before entering the air filtration system may be an effective way to reduce or eliminate any growth of microbes in the filtration system, or extend the life of the filtration system. The inlet air duct may be made out of a material that allows UV light transmission therethrough, such as a clear plastic or glass material. A separator 58 is configured under the UV light sources to capture any debris that might fall from the UV light sources.

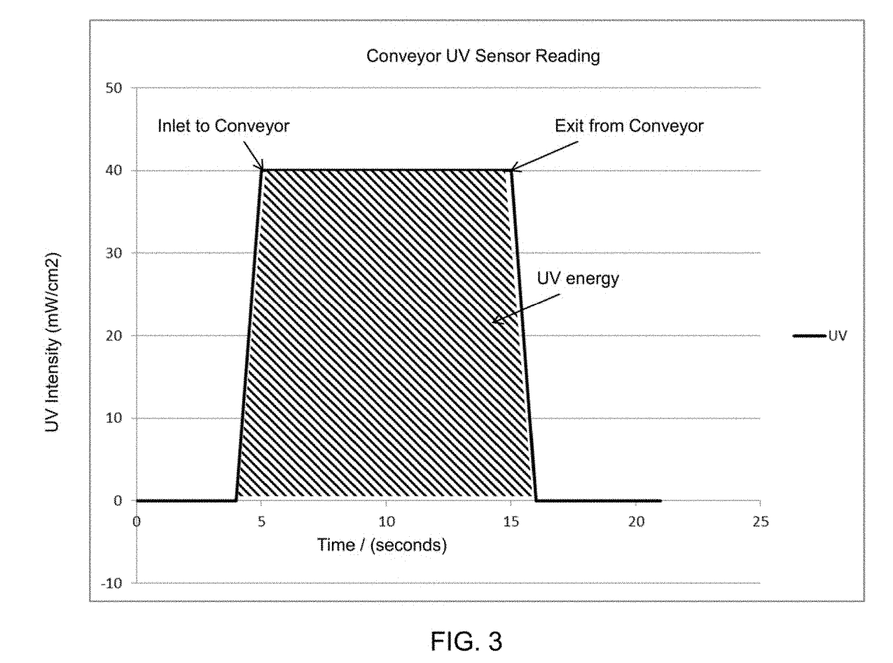

As shown in FIG. 2, an exemplary disinfecting conveyor system 10 comprises a conveyor belt 32 having a length 35 from the receiving end 34 to the delivery end 36. The conveyor belt has a top span 42 and a bottom span 44 and a drive 40 to spin the conveyor belt. The speed of the conveyor may be determined by a speed sensor 49. The conveyor belt has a top surface 37, a bottom surface 38 and a plurality of apertures 39 therethrough, from the top surface to the bottom surface. The apertures allow UV light to pass through to expose substantially the entire outer surface of the product 11 to UV light. The apertures enable the UV light to be incident on the product through the bottom surface of the top span 42 of the conveyor belt. Debris 17 from the product falls 17 down into the air exhaust vents 72 where it can be removed from the enclosure to prevent contamination. A UV light sensor 54 is configured to read a level of UV light with the enclosure and provide this reading to the control system. In addition, a conveyor UV light sensor 55 is coupled to the conveyor and moves through the enclosure on the conveyor. The conveyor UV light sensor provides a UV light reading as a function of time. As the conveyor UV light sensor 55 enters the enclosure 20 it has a rapid increase in the UV light reading and as the conveyor UV light sensor 55 exits the enclosure 20 it has a rapid decrease in the UV light reading, as shown in FIG. 3. The total UV light exposure can be calculated by the control system and used to increase the UV light intensity or the speed of the conveyor to ensure that an effective amount of UV light exposure is provided for a given type of product. The exemplary conveyor belt 32 comprises a belt tracking feature 150. A belt tracking sensor 142 senses the belt tracking feature and provides data to the control system 90 and the control system adjusts the position of the belt by a belt alignment feature 156, to keep it aligned. A belt alignment feature may slightly change the angle of one of the conveyor drives 40, for example. As shown in FIG. 2, product 11, such as a food product 12 is being conveyed along the belt on the top surface 37 and in the product portion 132 of the UV transmission belt and a UV light source 52″ under the belt projects UV light up onto the product.

As shown in FIG. 3, the output from a conveyor UV sensor enables the control system to calculate UV light exposure, or dose of UV light that a product passing through the disinfecting conveyor system receives. The UV light intensity mW/cm² and time are used to calculate the UV dose.

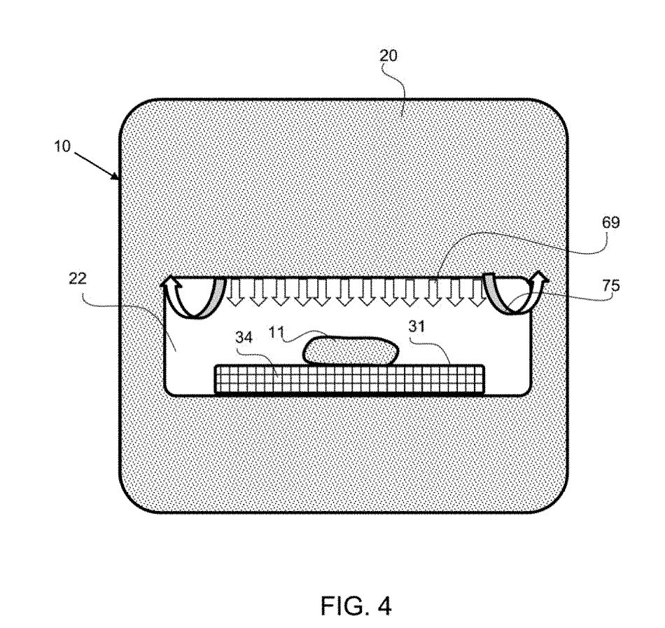

As shown in FIG. 4, an exemplary disinfecting conveyor system 10 has an inlet or an opening to the enclosure 20. The receiving end of the conveyor belt 34 has a product 11, disposed thereon for moving through the disinfecting conveyor system. A laminar air flow 69 within the enclosure keeps the product cool while the UV light disinfects the product. The enclosure has a positive exit air flow 75 from the inlet 22 opening and from the out opening to ensure no contamination enters into the enclosure.

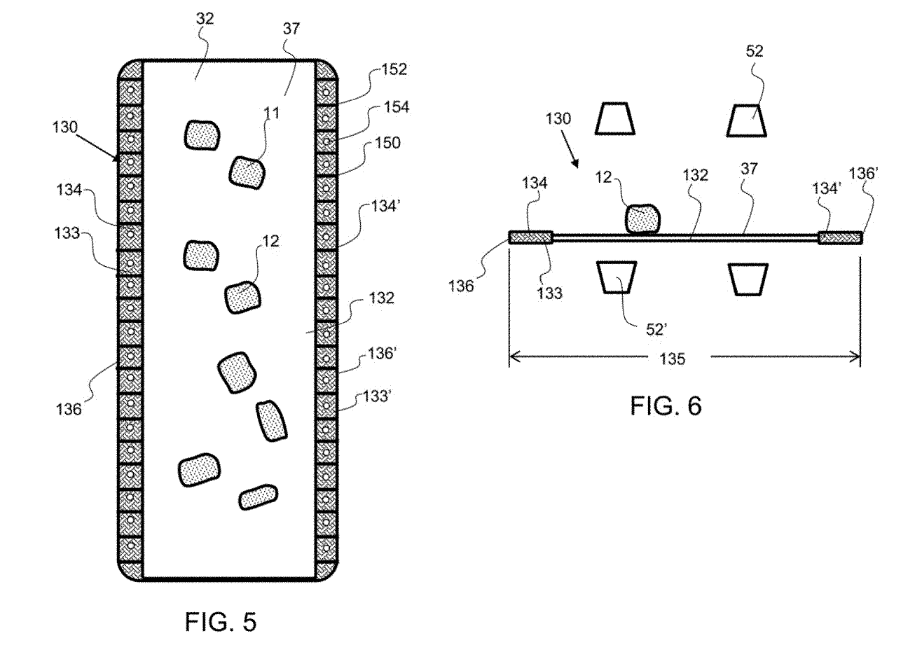

Referring now to FIGS. 5 and 6, an exemplary conveyor belt 32 is a UV transmission belt 130 having a product portion 132 that is at least UV translucent and an edge portion 134, 134′. The conveyor belt has a width 135 from a first edge belt edge 136 to an opposing belt edge 136′. The edge portion may comprise a support material 133 such as a folded portion of the conveyor belt that is retained in a folded configuration with adhesive, a heat weld, or a fastener, such as a stitch. An exemplary edge portion may be thicker than the product portion 132, as best shown in FIG. 6. As described herein, the edge portion may provide for better tracking of the belt and may reinforce the belt to prevent stretching or warping of the belt overtime. The edge portion of the conveyor belt or a product portion of said belt may be at least UV translucent wherein about 25% or more of the UV light passes through the belt and preferably about 50% or more and even more preferably about 85% or more. When the conveyor belt or a portion thereof, such as the edge or product portion has about 90% of more UV light transmission, it is UV transparent for the purposes of this invention. An exemplary belt may comprise a belt tracking feature 150, such as printed material 152 or holes 154, for example. A belt tracking sensor 142 may sense the belt tracking feature and provide data to the controller to adjust the position of the belt to keep it aligned as shown in FIG. 2. A product 11, such as a food product 12, is being conveyed along the belt on the top surface 37 and in the product portion 132 of the UV transmission belt and UV light sources 52 and 52′ are configured above and below the belt, respectively. In this way, the entire surface area of the product is exposed to UV light.

It will be apparent to those skilled in the art that various modifications, combinations and variations can be made in the present invention without departing from the spirit or scope of the invention. Specific embodiments, features and elements described herein may be modified, and/or combined in any suitable manner. Thus, it is intended that the present invention cover the modifications, combinations and variations of this invention provided they come within the scope of the appended claims and their equivalents.

What is claimed is:

1. A disinfecting conveyor system comprising:
a) a control system;
b) an enclosure comprising:
i) a length from an inlet end to an outlet end;
ii) an inlet;
iii) an outlet;
c) a conveyor belt comprising:
i) a receiving end and a delivery end;
ii) a top surface and a bottom surface; and
iii) a top span and a bottom span;
iv) a width from a first edge to a second edge;
v) a product portion extending between the first and second edges that is at least UV translucent having at least 50% UV light transmission therethrough;
wherein the conveyor extends along the length of the enclosure and is configured to move a product from the inlet to the outlet;
d) a UV light source configured above the top surface and top span of the UV transmission conveyor belt;
e) a UV light source configured under the top surface and top span of the UV transmission conveyor belt;
wherein the UV light sources are configured to project UV light onto said product as it moves through the enclosure;
wherein said plurality of UV light sources are configured to project light substantially 360 degrees about the product as it moves through the enclosure
f) an inlet air duct comprising a UV transparent material to allow the UV light produced by the plurality of UV light sources to pass therethrough,
wherein said inlet air duct extends within the enclosure and is exposed to the UV light from the plurality of UV light source; and
wherein the inlet air is at least partially disinfected by exposure to the UV light as it passes through the inlet air duct.

2. The disinfecting conveyor system of claim 1, further comprising an air cooling system configured to cool an inlet air flow to produce a cooled air flow for deliver as a process air into the enclosure.

3. The disinfecting conveyor system of claim 1, further comprising an air filtration system configured to remove particles from the inlet air flow and deliver a flow of clean air as said process air into the enclosure.

4. The disinfecting conveyor system of claim 3, wherein the filtration system meets HEPA efficiency, wherein 99.7% of said particles having a size of 0.3 um or greater are removed from the inlet air flow.

5. The disinfecting conveyor system of claim 3, wherein the process air is delivered into the enclosure by an inlet vent configured to produce a flow of process air down over a product as it passes through the enclosure.

6. The disinfecting conveyor system of claim 5, wherein the enclosure comprises an exhaust vent configured below the conveying surface of the conveyor belt, wherein the process air flows down from the inlet vents configured above the conveyor belt, over the product and down below the conveying surface of the conveyor belt and into the exhaust vent.

7. The disinfecting conveyor system of claim 6, wherein the belt comprises a plurality of apertures that extend through the belt from the top surface to the bottom surface to allow UV light to pass therethrough and onto the product carried thereon.

8. The disinfecting conveyor system of claim 7, wherein the belt has an open area of at least 75%.

9. The disinfecting conveyor system of claim 1, wherein the product portion of the conveyor belt is a plastic comprising at least one of Acetal homopolymer, Nylon, Polybutylene terephthalate (PBT), polyethylene (PE), high density polyethylene (HDPE), polyvinylidene fluoride (PVDF), polyetherimide (PEI), polyozymethylene (POM), fluoropolymers, Fluorinated ethylene propylene (FEP), perfluoroalkoxy alkane (PFA) or tetrafluoroethylene-hexafluoropropylene-vinylidene fluoride (THV).

10. A disinfecting conveyor system comprising:
a) a control system;
b) an enclosure comprising:
i) a length from an inlet end to an outlet end;
ii) an inlet;
iii) an outlet;
c) a conveyor belt comprising:
i) a receiving end and a delivery end;
ii) a top surface and a bottom surface; and
iii) a top span and a bottom span;
iv) a width from a first edge to a second edge;
v) a product portion extending between the first and second edges that is at least UV translucent having at least 50% UV light transmission therethrough;
wherein the conveyor extends along the length of the enclosure and is configured to move a product from the inlet to the outlet;
d) a UV light source configured above the top surface and top span of the UV transmission conveyor belt;
e) a UV light source configured under the top surface and top span of the UV transmission conveyor belt;
wherein the UV light sources are configured to project UV light onto said product as it moves through the enclosure;
wherein said plurality of UV light sources are configured to project light substantially 360 degrees about the product as it moves through the enclosure; and
f) a belt tracking system comprising a belt tracking sensor that senses a position of the belt and sends data to control system to adjust said position of said belt.

11. The disinfecting conveyor system of claim 10, comprising a UV light sensor coupled to the conveyor and configured to measure a measured UV intensity and provide said measured UV light intensity to the control system; wherein the control system monitors the measured UV light intensity and calculates a UV light exposure value for the disinfecting conveyor system.

12. The disinfecting conveyor system of claim 11, comprising a UV light sensor within the enclosure and configured to measure a measured UV light intensity and provide said measured UV light intensity to the control system

13. The disinfecting conveyor system of claim 11, wherein the control system receives a set point UV light intensity value and wherein the control system automatically changes the UV light intensity as a function of the measured UV light intensity and a set point UV light intensity.

14. The disinfecting conveyor system of claim 11 wherein the control system receives a set point UV light intensity value and wherein the control system will initiate an alarm, in the event that the measured UV light intensity is below said set point UV light intensity value.

15. The disinfecting conveyor system of claim 11, wherein the control system utilizes an exposure time of the UV light sensor along with the measure UV light intensity to calculate a UV light exposure value.

16. The disinfecting conveyor system of claim 15, wherein the control system determines exposure time from a change in measured UV light intensity.

17. A disinfecting conveyor system comprising:
a) a control system;
b) an enclosure comprising:
i) a length from an inlet end to an outlet end;
ii) an inlet;
iii) an outlet;
c) a conveyor belt comprising:
i) a receiving end and a delivery end;
ii) a top surface and a bottom surface; and
iii) a top span and a bottom span;
iv) a width from a first edge to a second edge;
v) an edge portion;
vi) a product portion extending between the first and second edge portions that is at least UV translucent having at least 50% UV light transmission therethrough;
wherein the conveyor extends along the length of the enclosure and is configured to move a product from the inlet to the outlet;
d) a UV light source configured above the top surface and top span of the UV transmission conveyor belt;
e) a UV light source configured under the top surface and top span of the UV transmission conveyor belt;
wherein the UV light sources are configured to project UV light onto said product as it moves through the enclosure;
wherein said plurality of UV light sources are configured to project light substantially 360 degrees about the product as it moves through the enclosure.

18. The disinfecting conveyor system of claim 17, wherein the edge portion is at least UV translucent, having at least 50% UV light transmission therethrough.

19. The disinfecting conveyor system of claim 17, wherein the edge portion comprises a folded portion of the belt.

20. The disinfecting conveyor system of claim 17, wherein the edge portion comprises a support material.

21. The disinfecting conveyor system of claim 20, wherein the support material is a fabric.
